(19) United States
(12) Patent Application Publication
(10) Pub. No.: US 2024/0295921 A1
(43) Pub. Date: Sep. 5, 2024

(54) SYSTEMS AND METHODS FOR SMART HEAD-MOUNTED DRUG DELIVERY TO AN EYE

(71) Applicant: TWENTY TWENTY THERAPEUTICS LLC, South San Francisco, CA (US)

(72) Inventors: Hojr Sedaghat Pisheh, South San Francisco, CA (US); Sam Kavusi, South San Francisco, CA (US)

(21) Appl. No.: 18/115,899

(22) Filed: Mar. 1, 2023

(51) Int. Cl. G06F 3/01; A61B 3/00

(52) U.S. Cl. CPC G06F 3/013; A61B 3/0025

(57) ABSTRACT

Described herein are systems and methods for delivery of a drug to an eye using a head mounted drug delivery system (e.g., smart eyeglasses). The head mounted drug delivery system can include an iris detection and tracking module to detect an iris of an eye and blinking of the eye; a drug delivery module to receive at least one signal from the iris detection and tracking module and to release a drug onto the eye; and a transmitter to communicate data wirelessly from the iris detection and tracking module and/or the drug delivery module. The drug delivery module includes: a reservoir to store the drug, a pump to propel the drug from the reservoir to at least one nozzle, and at least one nozzle to spray the drug onto the eye. A mobile device can wirelessly receive data from the iris detection and tracking module and/or the drug delivery module.

### TECHNICAL FIELD

The present disclosure relates to drug delivery to an eye, and, more specifically, to systems and methods for efficient delivery of a drug to the eye using a smart head-mounted drug delivery device.

### BACKGROUND

Glaucoma is the leading cause of irreversible blindness and refers to a group of chronic, progressive eye diseases that cause vision loss. Glaucoma currently has no cure, but early treatment can often stop the damage and protect vision from further deterioration. Prescription eye drops are the most common treatment for glaucoma. However, patients often do not fully adhere to prescribed schedules and/or dosages when applying the eyedrops. In fact, over 3 million Americans have been diagnosed with glaucoma, but only 30% of these patients meet the full criteria of adherence with the prescribed use of eyedrops. Patients can have difficulties applying the correct dosage of an eye drop into the eye. For example, patients can miss the eye, by blinking or incorrectly lining up the opening with the eye and apply to little, patients can squeeze the bottle too tight and apply too many drops at one time, or patients can touch the eye with the tip of the bottle, which can lead to infections. In other instances, patients may simply forget to apply eyedrops on schedule.

### SUMMARY

Provided herein are systems and methods for delivery of a drug to at least one eye using a smart head-mounted drug delivery device. The smart head-mounted drug delivery device can increase patient adherence while minimizing infections and enabling a doctor to receive a full picture of the adherence.

In one aspect, the present disclosure includes a system for head mounted drug delivery. A head-mounted drug delivery device (e.g., smart eyeglasses) can include: an iris detection module configured to detect an iris of at least one eye of a user; a drug delivery module configured to receive at least one signal from the iris detection module and to release a drug into the user's eye, wherein the drug delivery module comprises: a reservoir configured to store the drug, wherein the reservoir is at least one of refillable and removable, a pump configured to propel an amount of the drug from the reservoir to at least one nozzle, and the at least one nozzle configured to spray the amount of the drug onto the user's eye; and a transmitter configured to communicate data wirelessly from the iris detection module and/or the drug delivery module. The mobile device can be configured to receive data from the iris detection module and/or the drug delivery module wirelessly.

In another aspect, the present disclosure includes a method for delivery of a drug to at least one eye using a smart head mounted drug delivery device. The method can include detecting, by an iris detection module of the head mounted drug delivery system, that at least one eye of the user is open; applying, by a drug delivery module of the head mounted drug delivery system, an amount of a drug to the at least one eye of the user; and sending, by a wireless transmitter of the head mounted drug delivery system, a report of the amount of the drug applied to the at least one eye of the user to a mobile device. The mobile device can log the amount of the drug applied to the at least one eye of the user.

In a further aspect, the present disclosure includes smart eyeglasses that can be used as the head mounted drug delivery system for drug delivery. The smart eyeglasses can include an iris detection module, comprising a light source and/or a detector, configured to detect an iris of at least one eye of a user, wherein the iris detection module is at least partially positioned on a frame of the eyeglasses. The smart eyeglasses can also include a drug delivery module coupled to the frame of the eyeglasses and configured to receive at least one signal from the iris detection module and to release a drug into the at least one eye of the user. The drug delivery module can include: a reservoir configured to store the drug, wherein the reservoir is at least one of refillable and removable; a pump configured to propel an amount of the drug from the reservoir to at least one nozzle; and the at least one nozzle configured to spray the amount of the drug onto the at least one eye of the user.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a head-mounted drug delivery system;

FIG. 2 is a diagram showing an example of the iris tracking module of FIG. 1;

FIG. 3 is a diagram showing an example of the drug delivery module of FIG. 1;

FIG. 4 is a diagram showing an example of the system of FIG. 1 implemented on a pair of eyeglasses;

FIGS. 5-6 are diagrams showing operation of the eyeglasses of FIG. 4;

FIG. 7 is a process flow diagram of an example method for tracking a user's use of a drug to increase adherence to a prescription;

FIG. 8 is a process flow diagram of an example method for drug delivery; and

FIG. 9 is a process flow diagram of an example method for logging a user's use of a drug.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “drug delivery system” refers to one or more technologies that deliver a drug to at least a portion of a patient's body.

As used herein, the term “head-mounted drug delivery system” refers to one or more drug delivery technologies that can be integrated within a device worn on a user's head. For example, a head-mounted system can be integrated into eyeglasses, mounted on a head-worn device (e.g., helmet, hat, visor, etc.), or the like to operate as a drug delivery system to deliver a drug into/onto a patient's eye. In some instances, the head-mounted drug delivery system can be configured to deliver drugs, traditionally applied as eyedrops, into a user's eye.

As used herein, the term “smart” refers to an electronic device that includes or is linked to one or more components (e.g., a processor) that facilitates one or more active processes (e.g., to perform at least one independent action). A smart electronic device can generally connect to one or more other electronic device or networks via one or more wireless protocols and can operate to some extent interactively and/or autonomously from the one or more other electronic devices. For example, eyeglasses that are referred to as “smart” can be used for drug delivery and tracking applications.

As used herein, the term “eyeglasses” has a similar meaning to the terms “spectacle” and “glasses” and refers to one or more lenses mounted in a frame that holds the one or more lenses in front of a person's eye or eyes and has at least one arm, typically two arms, that each extend over an ear of a wearer. Eyeglasses can include at least one of glasses for correcting or treating defective eyesight (e.g., near-sightedness, far-sightedness, astigmatism, or the like), sunglasses, safety glasses, and glasses used for merely aesthetic purposes.

As used herein, the term “frame” refers to a device and/or mechanism that is designed to hold the one or more lens in a proper position on a person's head such that the one or more lens is held in front of the person's eye(s). Frames can exist in a variety of styles, sizes, materials, shapes, and colors. Typically, the frame includes at least a bridge over the nose, rims around at least a portion of each of the one or more lens (typically two lenses) and holding the one or more lens to the frame, and hinged arms (or temples/temple pieces) that extend from an axial portion of the rims to temple tips that rest over the person's ears.

As used herein, the term “lens” refers to a generally clear device (but may include tinting, in some instances) held over one or more of a person's eyes by a frame. The lens can be, but is not limited to, a glass material, a plastic material, or the like that is substantially transparent to at least the visible light spectrum. The lens can be a prescription and/or corrective lens, a cosmetic lens, a sunglasses lens, a safety lens, or the like. For example, the lens can concentrate or disperse light rays.

As used herein, the term “drug” refers to a medicine, pharmaceutical, or other substance, such as saline, that has a biological (physiological, psychological, or the like) effect when introduced to at least a portion of the body. A drug can be used to treat, cure, prevent, alleviate, or diagnose a disease, symptom and/or disorder or to promote well-being. A drug can be in any state of matter (e.g., liquid, solid, gas), and may have one state for storage and another for application. One or more drugs may be combinable and/or dilutable.

As used herein, the term “eyedrop” refers to a liquid drug that can be topically applied to a user's eye. Eye drops can be prescription, over the counter, or non-controlled substances. In some instances, the liquid drug can include an amount of a solid drug suspended in an amount of a liquid, such as ultrapure water, a buffer, or the like.

As herein, the term “prescription” refers to an instruction from a medical practitioner to use a prescription drug, a non-prescription drug, or the like. As an example, the prescription can have an amount, dosage, frequency, schedule, and/or the like for administration of a drug to a user. In some instances, a prescription can refer to instructions from a medical practitioner for a “prescription drug” or pharmaceutical that can only be dispensed by a pharmacy on the medical practitioner's instruction. In other instances, a prescription can refer to the schedule prescribed by the medical practitioner, but the drug may be an over the counter or non-controlled substance such as saline eyedrops.

As used herein, the terms “patient”, “subject”, “user”, and the like can be used interchangeably and can refer to an animal (e.g., a human) suffering from a condition that can be treated with topical application of a drug to an eye (e.g., via eyedrops).

### II. Overview

The irreversible blindness caused by glaucoma may be slowed, or even effectively stopped, with early treatment, often with prescription eye drops. However, patients often are non-compliant with the prescribed schedules and/or dosages when applying the eyedrops. Compliance can be based on (1) whether the patient accepts the diagnosis that requires treatment, (2) whether the patient continues to take the medication for the duration of the instructed time period, and (3) whether the patient can properly execute the administration of the eyedrop. Studies reveal that even patients with the best of intentions satisfying criteria (1) and (2) often fail criteria (3) to properly administer medications in eyedrop form. Many patients inadvertently miss the eye (e.g., due to blinking at the wrong time or poor accuracy) and/or apply an incorrect amount of medication (too little or too much) without even realizing. In fact, a patient with poorer vision is generally the patient who needs the medication the most, but generally has the most trouble with administering the medication as instructed. Additionally, patients with mobility issues, such as a palsy or grip issues, also have a difficult time self-administering medications in the form of eyedrops.

Described herein are systems and methods for efficient delivery of a drug (e.g., traditionally delivered in the form of eyedrops) to the eye using a smart head-mounted drug delivery device. The systems and methods can increase adherence of use of eye medications, such as prescription or saline eyedrops and can more efficiently apply a drug to at least one eye in a manner that is more efficacious and minimizes drug waste. Additionally, the systems and methods can track patient adherence to provide physicians with a clear picture of patient compliance with a prescribed regimen and whether a treatment is working.

### III. System

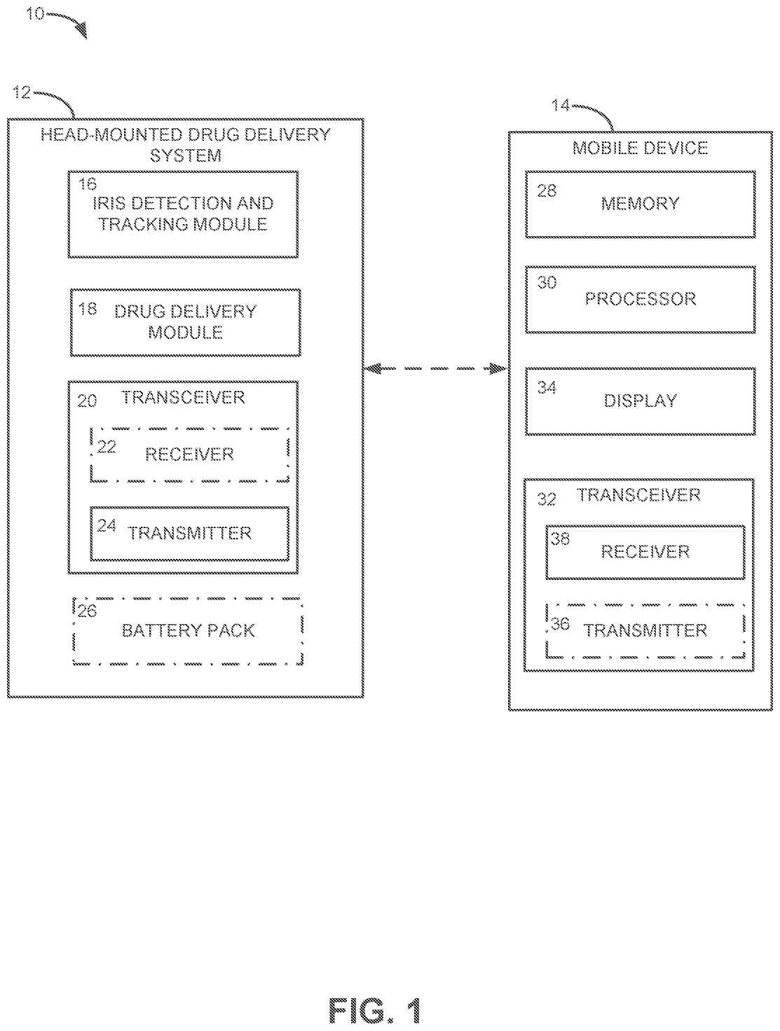

Provided herein is a system 10 (FIG. 1) for efficient delivery of a drug to a user's eye using a smart head-mounted drug delivery device. The drug can be in the form of eyedrops. An example of the smart head-mounted drug delivery device is the head-mounted drug delivery system 12. The head-mounted drug delivery system 12 can take the form of eyeglasses, but it should be understood that the head-mounted drug delivery system 12 can take the form of any head-mounted device that can deliver a drug to at least one of a user's eyes (e.g., a hat, a visor, or the like). The head-mounted drug delivery system 12 can deliver an amount of a drug to at least one eye of a user. The amount of the drug delivered to the at least one eye of a user can be at least one dose of the drug defined based on a prescription of the user, the FDA, and/or drug packaging instructions. The delivery of the drug can be manually controlled by the user (e.g., the user can trigger a time the drug is delivered via a button or an application on a mobile device or the like) and/or automatically controlled (e.g., the delivery can be set for predefined times based on a prescription or a packaging instruction or the like, or the delivery can be triggered based on one or more changes in a physiological variable of the patient).

The head-mounted drug delivery system 12 of the system 10 can increase the adherence of patients to taking a proper amount of a drug as needed and/or on a predefined schedule, while minimizing waste of the drug (e.g., from improper application). The system 10 also can include at least one mobile device (shown as mobile device 14) in wireless communication with the head-mounted drug delivery system 12. The mobile device 14 can communicate instructions (e.g., drug amounts to be delivered, predefined application times, manual delivery instructions, protocol changes, or the like) to the head-mounted drug delivery system 12 and can receive data (e.g., data about the drug delivery timing, amount used, amount saved, blinking timing information, or the like) from the head-mounted drug delivery system. The wireless communication can be at least between a transmitter 24 of the head-mounted drug delivery system 12 and a receiver 38 of the mobile device 14. In the other direction the wireless communication can be at least between a transmitter 36 of the mobile device 14 and a receiver 22 of the head-mounted drug delivery system 12. In some instances, the head-mounted drug delivery system 12 can include a transceiver 20 that can include both the receiver 22 and the transmitter 24 and the mobile device 14 can include a transceiver 32 that can include the transmitter 26 and the receiver 38 for bi-directional communication. In some instances, the wireless communication can be short-range wireless communication, using Bluetooth, infrared, ultraband, Zigbee, RF, or the like. In some instances, the short-range wireless communication can use RF.

As an example, data communicated to the mobile device 14 from the head-mounted drug delivery system 12 can include information that can give a medical professional (e.g., a physician, an optometrist, ophthalmologist, nurse, or the like) a clear picture of patient compliance with a prescribed drug regimen (e.g., amounts of drug applied, times drug applied, if drug applied as scheduled, iris information, blinking information, or the like) to aid in the decision of whether a treatment with the drug is working to treat the patient. In another example, data communicated to the head-mounted drug delivery system 12 from the mobile device 14 can include, but is not limited to, a trigger from the user for a manual drug release by the head-mounted drug delivery system 12 and/or instructions (e.g., from a medical professional) for altering one or more amounts of a drug to be delivered, scheduled timings, and or automatic trigger thresholds for applying the drug.

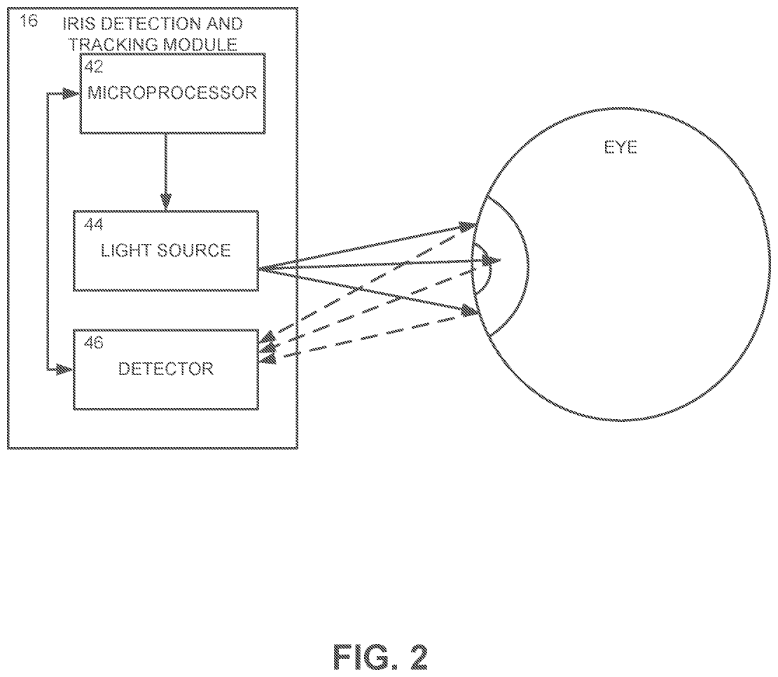

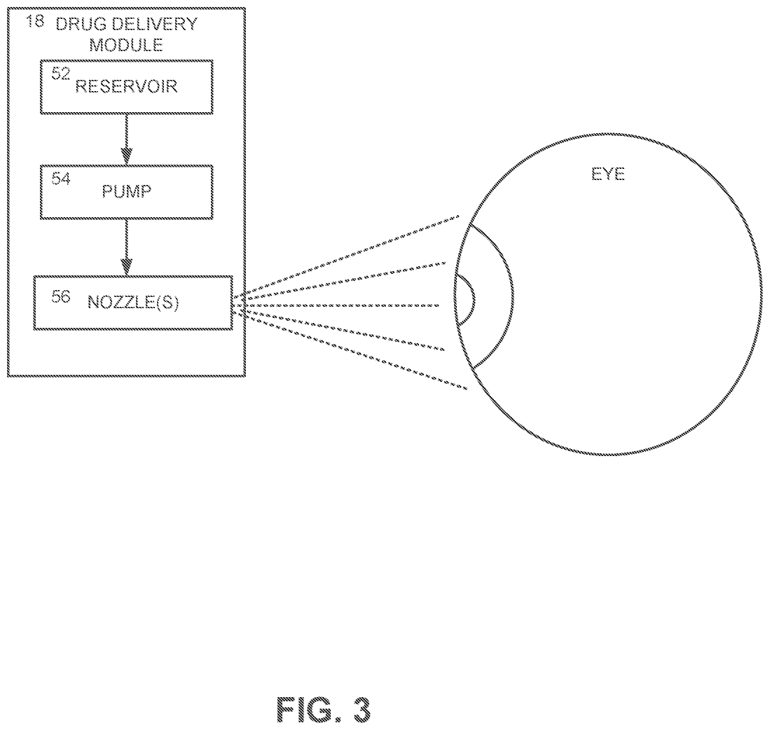

The head-mounted drug delivery system 12 can efficiently deliver the amount of the drug to the at least one eye of the patient with less drug waste. The head-mounted drug delivery system 12 can include an iris detection and tracking module 16 (shown in more detail in FIG. 2) that can detect an iris of at least one eye of the user and, in some instances, track the detected iris. For example, the iris detection and tracking module 16 can track blinking of the user, determine if the head-mounted drug delivery system 12 is worn by the correct user and/or positioned correctly to deliver a drug to the at least one eye of the user. The head-mounted drug delivery system 12 can also include a drug delivery module 18 (shown in more detail in FIG. 3) that can receive at least one signal from the iris detection and tracking module 16 and release a drug towards at least one of the user's eyes. For example, the drug delivery module 18 can deliver an amount of a drug towards or to at least one eye of the user as a spray of a liquid drug. The iris detection and tracking module 16 and the drug delivery module 18 can be communicatively connected to each other and any of the other components of the head-mounted drug delivery system 12 with any combination of a wired and/or a wireless connection.

As previously mentioned, the head-mounted drug delivery system 12 can also include a transceiver 20 that can include a transmitter 24 and, in some instances, receiver 22 for bi-directional communication. It should be noted that in some instances, the head-mounted drug delivery system 12 includes transmitter 24, but the receiver 22 is optional (and aids in bidirectional communication). The transmitter 24 and the, optional, receiver 22 may alternatively be embodied as separate device elements. The transceiver 20 (or the transmitter 24 and/or receiver 22) can communicate data wirelessly between the head-mounted drug delivery system 12 and the mobile device 14 such that data from the iris detection and tracking module 16 and/or the drug delivery module 18 can be sent to the mobile device. The mobile device 14 can receive the data from the iris detection and tracking module 16 and/or the drug delivery module 18 wirelessly (as previously noted). Additionally, the head-mounted drug delivery system 12 can include a power source (shown as battery pack 26) that can power at least the iris detection and tracking module 16, the transceiver 20, and/or the drug delivery module 18. The battery pack 26 can be rechargeable and/or receive line power. The head mounted drug delivery system 12 can include additional components (not shown) like an ambient light sensor that can enable the iris detection and tracking module 16 to measure pupil size change of the at least one eye of the user, for example.

Referring again to the mobile device 14, the mobile device can collect statistics related to the user's compliance with using the head mounted drug delivery system 12 and/or statistics related to the drug delivery based on the data from the iris detection and tracking module 16 and the drug delivery module 18. The mobile device can be, for example, a smart phone, a tablet computer, a lap top computer, or the like, or a specially designed device for communicating with the head-mounted drug delivery system 12. For example, the mobile device 14 can run an application that controls and logs delivery of the drug, delivery amount of the drug, delivery date/time, or the like for the patient/doctor.

The mobile device 14 can include a memory 28 that can store instructions and data and a processor 30 that can execute the instructions to cause the performance of operations. The memory 28 can be a non-transitory memory device (e.g., RAM, ROM, solid state device, etc.). In some instances, the processor 30 and memory 28 can be part of the same device (e.g., a microprocessor). In other instances, the processor 30 and memory 28 can be separate devices. The mobile device 14 can also include a display 34 that can provide data, notifications, instructions, alerts and/or manipulated data, or the like to a user (e.g., in visual and/or audio form). The mobile device 14 can include a transceiver 32 with a receiver 38 and, in some instances, a transmitter 26. Alternatively, the receiver 38 and the, optional, transmitter 26 can be separate devices (not shown). It should be noted that, in some instances, the mobile device 14 includes receiver 38, but the transmitter 36 is optional (and aids in bidirectional communication).

As an example of the iris detection and tracking module 16 is shown in further detail in FIG. 2. The iris detection and tracking module 16 can detect parts and/or all the iris of at least one eye of the user. One iris detection and tracking module 16 may detect an iris of one eye or the iris of each eye of the user. For example, the iris detection and tracking module 16 can detect presence, movement, location, and/or occlusion of at least part of the iris. In other instances, the iris detection and tracking module 16 can also track the iris. For example, the iris detection and tracking module 16 can track blinking speeds, gaze direction, and the like. Based on the detection and/or tracking of at least part of the iris the iris detection and tracking module 16 can determine whether the correct user is wearing the head-mounted drug delivery system 12, whether the head-mounted drug delivery system is worn correctly, whether the user has blinked, a speed of the blink, or the like. In some instances, the iris detection and tracking module can have structure/functionality similar to modules used in AR/VR platforms.

The iris detection and tracking module 16 can include at least one light source (shown as light source 44) and at least one detector (shown as detector 46) that work together to perform the functionality of the iris detection and tracking module 16. The iris detection and tracking module 16 can also include a microprocessor 42 that can send instructions to the light source 44 and/or the detector 46 and receive data from the detector. As shown on the right side of FIG. 2, the at least one light source 44 can emit a light toward at least one eye of the user and the at least one detector can receive reflected light based on the light emitted towards the at least one eye. For example, the detector 42 can detect a greater amount of reflected light when the eye is open than when the eye is shut (e.g., the eye is more reflective than the eyelid. In other instances, specific portions of the eye may have different reflectance than others so the detector can detect different amounts, intensities, wavelengths, or the like based on where the light reflected on the eye (e.g., iris, pupil, sclera, etc.). The detector 46 can format the reflected light into data that can be sent to and read by the microprocessor 42 of the iris detection and tracking module 16 and/or to the mobile device 14. While not shown, the iris detection and tracking module 16 can also include any necessary motors, lenses, mirrors, or other components necessary for the emitting, receiving, and detecting of light signals.

As an example, the at least one light source 44 can include an infrared (IR) light source (to provide IR light that does not damage the eye) and the at least one detector 46 can include a camera (e.g., a camera configured to take photographs at discrete times, a video recorder configured to record a video for continuous times (or rates considered continuous), etc.) or a photodetector. For example, the detector 42 can be configured to detect whether the head mounted drug delivery system 12 is worn by the user (e.g., on a person's head vs. sitting on a surface or in a case) and the iris detection and tracking module 16 and/or the mobile device associated with the system 10 can be configured to authenticate the user wearing the head mounted drug delivery system based on the detection by the camera so only the user meant to receive the drug receives the drug. In some instances, features of the iris of the user can be detected by the camera and compared to features of the iris of the authenticated user stored in a memory (in either the microprocessor 42 of the iris detection and tracking model 16 or the mobile device 14) during a set-up process. The mobile device can be configured to notify and/or alert the user when the iris detection and tracking module 16 and/or the mobile device 14 determines the user should be wearing the head-mounted drug delivery system 12 (e.g., scheduled drug delivery time is imminent), the user is improperly wearing of the head-mounted drug delivery system, a non-authenticated user is wearing the head-mounted drug delivery system, and/or the user is non-compliant with instructions.

In another example, the iris detection and tracking module 16 can detect whether the user has blinked, and/or the user's blink speed over a given time period) and send a signal to the drug delivery module (e.g., drug delivery module 18) to deliver an amount of a drug toward at least one eye of the user at a time after the user has blinked. The delivery of the drug can be timed after a blink to ensure the drug is delivered to the eye in the correct amount (e.g., a therapeutic and/or prescribed amount) rather than a closed eye lid. The iris detection and tracking module 16 can also determine the head-mounted drug delivery device 12 is worn correctly so that the drug delivery module is lined up emit the drug towards the eye (so not too high, low, right, or left). In both of these ways user compliance with instructions for the eye to receive a certain amount of a drug can be improved simply through wearing the head-mounted drug delivery system 12. The detector 46 and/or the entire iris detection and tracking module can also be configured for other purposes, such as to detect dry eye and/or detect changes in intraocular pressure based on changes in a surface of the cornea.

The microprocessor 42 can be linked to at least one light source 44 and at least one detector 46 to provide instructions and/or receive data. The microprocessor 42 can include the functionality of a memory and a processor as described above. The microprocessor 42 can instruct the at least one light source 44 to emit light towards at least one eye of the user and the at least one detector 46 to detect light reflected from the at least one eye of the user (and send data based on the reflected light to the microprocessor 42). In some instances, the microprocessor can send parameters of the light to be emitted to the light source 44, such as, but not limited to, intensity, wavelength(s), direction, pulse, beam size, or the like. The microprocessor 42 can process the data received from the at least one detector 46 to determine a state of the eye (e.g., open or closed, correctly focused, disease state, or the like) and/or can send the data to at least one mobile device for further processing.

An example of the drug delivery module 18 is shown in greater detail in FIG. 3. The drug delivery module 18 can include at least a reservoir 52, a pump 54, and one or more nozzle(s) 56. The drug delivery module 18 and its component parts can be any shapes and/or sizes that can deliver an amount of at least one drug to at least one eye of a user, for example, the drug delivery module 18 can be shaped as or built into a housing shaped as a part of a frame of a pair of eyeglasses. The reservoir 52 can store a volume of a drug that includes multiple dosages (e.g., 0.5 mL, 1 mL, 5 mL, 10, 20 mL, or the like). The reservoir 52 can be refillable and/or removable (e.g., for exchange when the reservoir is empty, when a medical professional makes a change to the drug prescribed, or the like). The pump 54 can be in fluid communication with the reservoir and can propel an amount of the drug from the reservoir 52 to the one or more nozzle(s) 56. The pump 54 and the one or more nozzles 56 can be connected with at least one tube that can facilitate the transport of the drug from the reservoir to the one or more nozzles. The one or more nozzle(s) 56 can control and direct the drug toward the eye and spray the amount of the drug into and/or onto one of the user's eyes (example shown on the right side of FIG. 3). The one or more nozzles 56 can include one or more openings. The drug delivery module 18 can activate to deliver an amount of the drug to at least one eye of the patient based on instructions received from the microprocessor of the iris detection and tracking module and/or the mobile device. The drug delivery module 18 in combination with the iris detection and tracking module can time the delivery of the drug so that the eye is open to receive the drug. In some instances, not shown, the one or more nozzle(s) 56 can include a drop absorption mechanism below at least a portion of the one or more nozzles that can catch any remaining droplets of the drug from at least one opening of the nozzle and send the remaining droplets of the drug back to the reservoir 52.

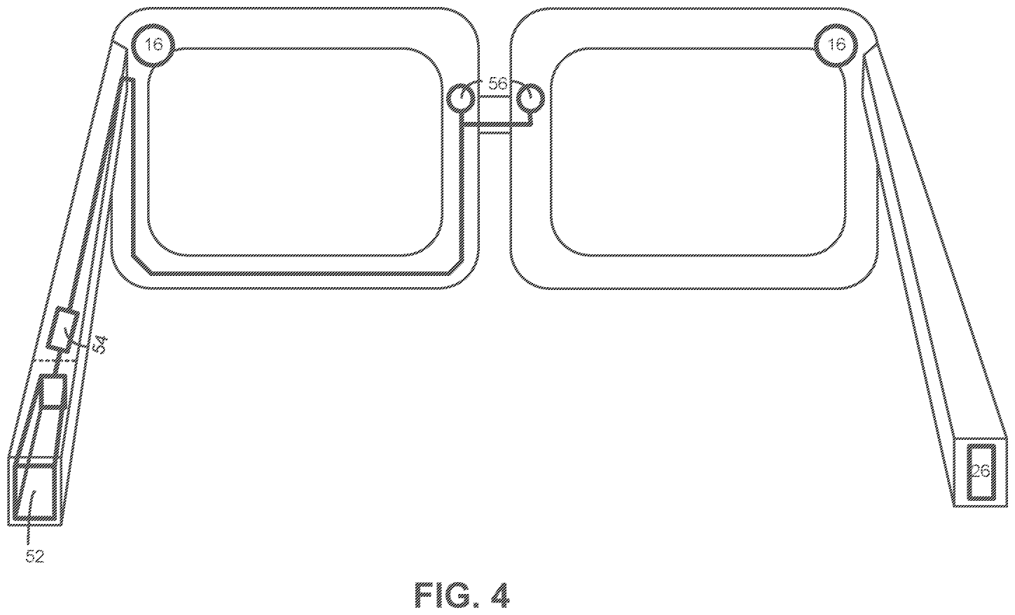

FIG. 4 is a diagram showing elements of the head-mounted drug delivery system 12 implemented on and/or in a pair of eyeglasses with lenses and a frame. It should be noted that lenses can be prescription, non-prescription, or merely aesthetic and may not be required. Additionally, the frame as shown is merely representative and can be any shape, material, or design of eyeglasses frame. The head-mounted drug delivery system 12 can be partially or fully manufactured as a pair of eyeglasses or retroactively added to a pair of eyeglasses that already exist (e.g., through mechanical attachments, adhesives, or the like). The iris detection and tracking module 16 can be located near at least one of the temples of the user, as shown in FIG. 4 an iris detection and track module 16 is located near both the right and left temples and substantially on the frame of the eyeglasses. The iris detection and tracking module 16 can be located at least in part on, in, or near a part of the frame and/or can be located on, in, or near a lens such that the at least one light source (e.g., light source 44) is positioned to emit light toward the eye.

Components of the drug delivery module (e.g., drug delivery module 18), including the reservoir 52, the pump 54, intervening tubing (not numbered), and two nozzles 56 are shown in FIG. 4. One or more components of the drug delivery module can be embedded in at least one part of the eyeglasses or attached to the eyeglasses. In FIG. 4, the reservoir 52 is shown inside an arm of the frame of the eyeglasses that can be opened (e.g., shown as a hinge via a dotted line, but can be any opening mechanism) to replace or refill the reservoir. The pump 54 is also shown inside the arm and connected to the reservoir via tubing (also inside the arm). It should be understood that the pump 54 can also be in direct attachment with the reservoir 54 or at any other location in fluid communication between the reservoir and the one or more nozzle(s) 56. One or more pumps 54 may be included. Two nozzles 56 are shown on either side of a nose bridge of the eyeglasses and such that each nozzle directs the drug to a single eye, but any number of nozzles one or more and positioned to effectively direct the drug to at least one eye can be included. The nozzle(s) 56 can be partially embedded in the frame or attached on the frame on the user facing side of the eyeglasses to spray the drug toward at least one eye. The battery pack 26, including any other circuitry, such as a transmitter, receiver, and/or transceiver, is also located on/within the frame and can be configured to power the iris detection and tracking module 16 and/or one or more portions of the drug delivery module (e.g., drug delivery module 18). The components can be configured and connected as shown in FIG. 4. However, it should be understood that FIG. 4 is only an example and other configurations and connections are possible.

The data collected by the iris detection and tracking module 16 can be used to monitor at least one of eye detection, eye tracking, and blinking as described previously. For example, the light source and/or the camera of the iris detection and tracking module 16 can detect at least one eye of the user blinking and then send a signal to the drug delivery module (e.g., drug delivery module 18) to deliver the drug after a blink has ended. This can occur at a scheduled time, in response to a manual trigger from the user, or in response to a physiological input (e.g., from a sensor in communication with the head-mounted drug delivery system, such as an intraocular pressure sensor or tear film measuring sensor) passing a predefined threshold. The drug delivery module (e.g., drug delivery module 18) can receive the signal and deliver a predetermined amount of the drug to at least one eye of the user at a time after the user has blinked.

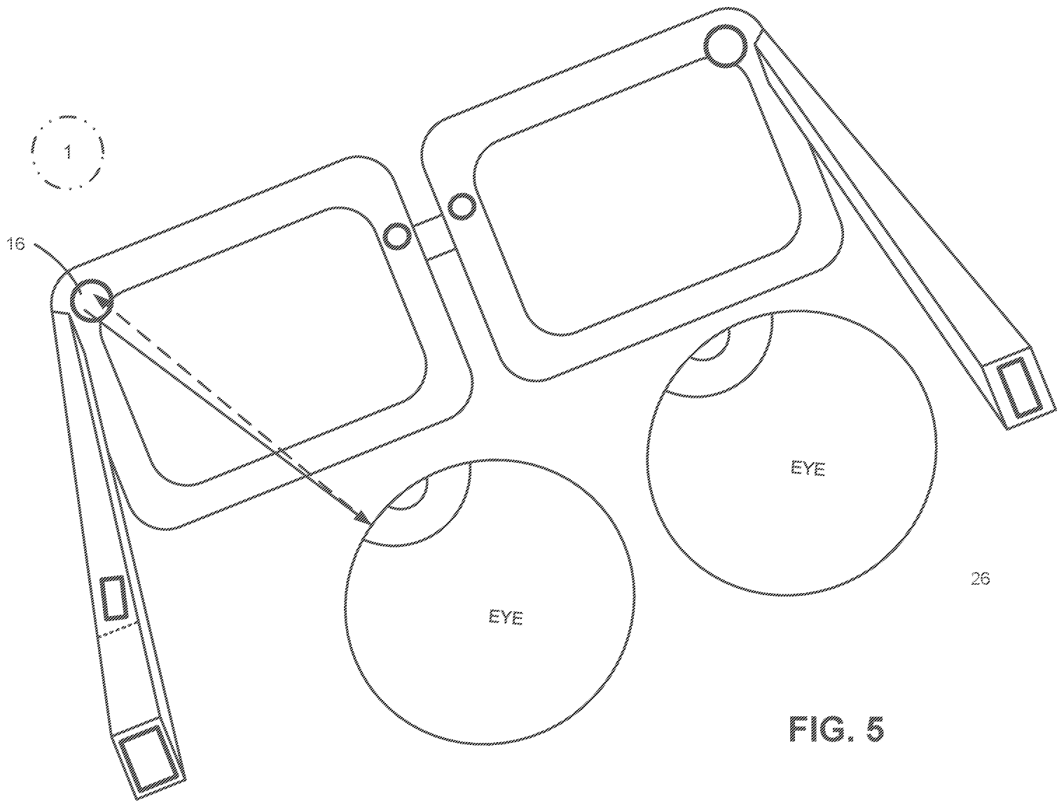

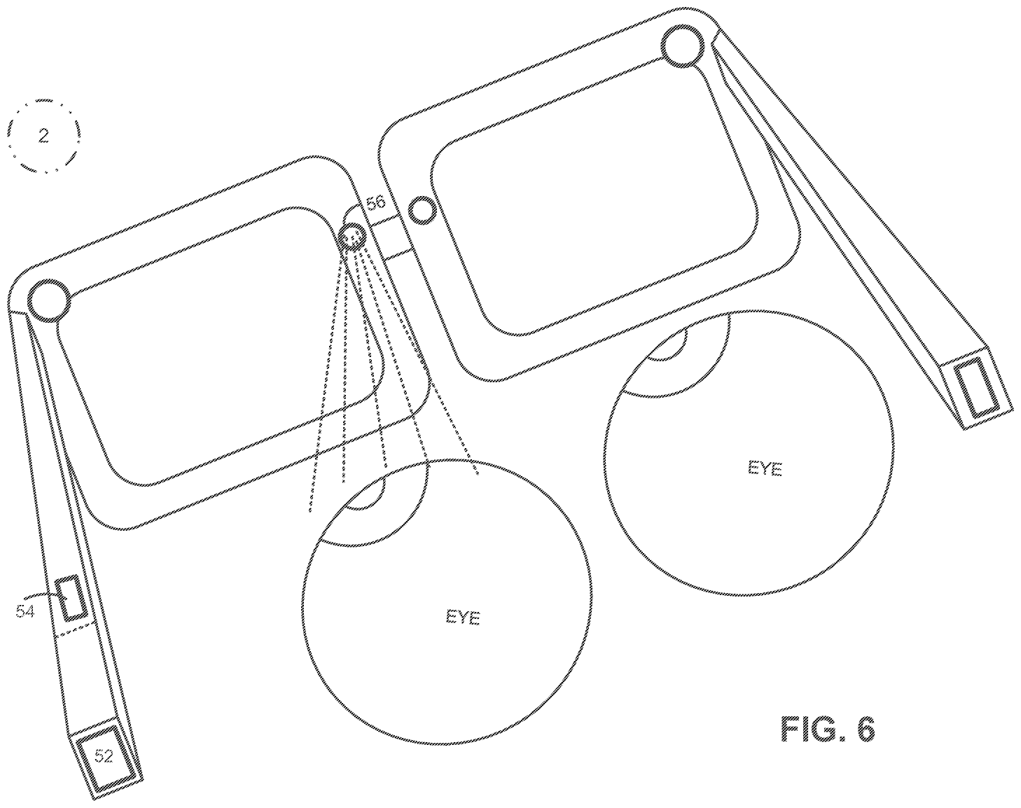

FIGS. 5-6 are diagrams showing operation of the eyeglasses example of the head-mounted drug delivery system 12 of FIG. 4 (the entirety of the connections and numbering mirror FIG. 4 but are not shown for simplicity of illustration) with reference to a single eye for simplicity of illustration. It should be understood that corresponding components can function on the other eye. FIG. 5 shows (1) an example of the iris detection and tracking system 16 emitting light (solid line) towards the iris (but can be towards any visible part of the eye (e.g., sclera, pupil, etc.), or the eyelid if the lids are partially or fully closed) and detecting reflected light (dashed) from the iris (but can be any visible part of the eye (e.g., sclera, pupil, etc.) or the eyelid if the lids are partially or fully closed). FIG. 6 shows (2) an example of the drug delivery module 18 (with the reservoir 2, pump 44, and nozzle 46) delivering an amount of the drug (dotted lines) to an eye of the patient. It should be noted that the eyeglasses example of the head-mounted drug delivery system 12 shown in FIGS. 4-6 can communicate with the mobile device of FIG. 1 (although not illustrated in FIGS. 4-6) as described with respect to FIGS. 1-3. One or more of the components of the head-mounted delivery system 12 can be programmable by the mobile device.

The iris detection and tracking module of the eyeglasses shown in FIGS. 5 and 6 can include an IR light source that provides IR light (but can be a light source that emits one or more lights at wavelengths that do not harm the eye) and a camera (or other detector) using motion tracking and/or blink tracking software, recognition software, or software for detecting one or more ailments or disorders of the eye (e.g., within a microprocessor of the iris detection and tracking module, within an external mobile device (not illustrated), and/or the like). In some instances, the iris detection and tracking module can be used for iris recognition to authenticate the person wearing the eyeglasses is the user meant to receive a treatment. As shown in FIG. 5, the iris detection and tracking module can emit a beam of IR light towards the iris of an eye that can be reflected and the reflected light can be detected by the camera to track whether the eye is open or blinking. Blinks can be detected using one or more eye closed segmentation techniques and/or machine learning techniques without requiring a custom controller (and, in some instances, without requiring eye tracking). In other words, the camera can monitor the blinking (continuously and/or only for a period of time prior to drug delivery) and the machine learning algorithm can be used to find the best time to deliver the drug into the eye. Additionally or alternatively, the iris detection and tracking module can be used to detect dry eye, detect changes in internal eye pressure (e.g., intraocular pressure) based on the detectable changes in the surface of the cornea, measure pupil size change in response to ambient light (detected by an ambient light sensor), or the like.

A time after a blink while the eyelids are open, the drug delivery module of FIGS. 5 and 6 can spray a drug into an eye of the user (shown in FIG. 6). As an example, the drug can be an eyedrop that is similar to an artificial tear and/or a drug that can be used in the treatment of glaucoma. The reservoir of the drug delivery module can at least store an amount of the drug that allows a user to apply the drug multiple times a day, a few days, a week, or up to a month (or more) based on, for example, a schedule assigned by a medical professional. In some instances, the glasses and/or the mobile device (not shown) can alert the patient when the drug needs to be refilled. The reservoir can include a cartridge that can be small and designed to minimize contamination and bacteria growth and easily replaceable. Reservoirs and/or cartridges can be designed to hold multiple drugs, in some instances. As shown in FIGS. 4-6, the nozzle(s) can be positioned with respect to the eye such that the direction of the drug spray is substantially centered on an eye. Additionally, each nozzle can include a single nozzle, with one or more openings, or a plurality of small nozzles such that the nozzle(s) each deliver nanoliter volume droplets of a drug for more accurate drug delivery (because large droplets tend to come out of the eye when the patient blinks). When two nozzles are used, each on a side (left or right) of eyeglasses, then each side can have the same nozzle or different nozzles, such that the left side can have a single nozzle, but the right side can have a plurality of small nozzles. Each nozzle can have a drop absorber/channel integrated to sink remaining droplet from the nozzle after delivery of the drug to increase the efficient drug use and minimize drug waste. It should be noted that a smaller quantity of the drug may be delivered to an eye because more of the drug can make it to the eye, the drug can be applied more frequently (because delivery is simpler for the user) or the like.

### V. Method

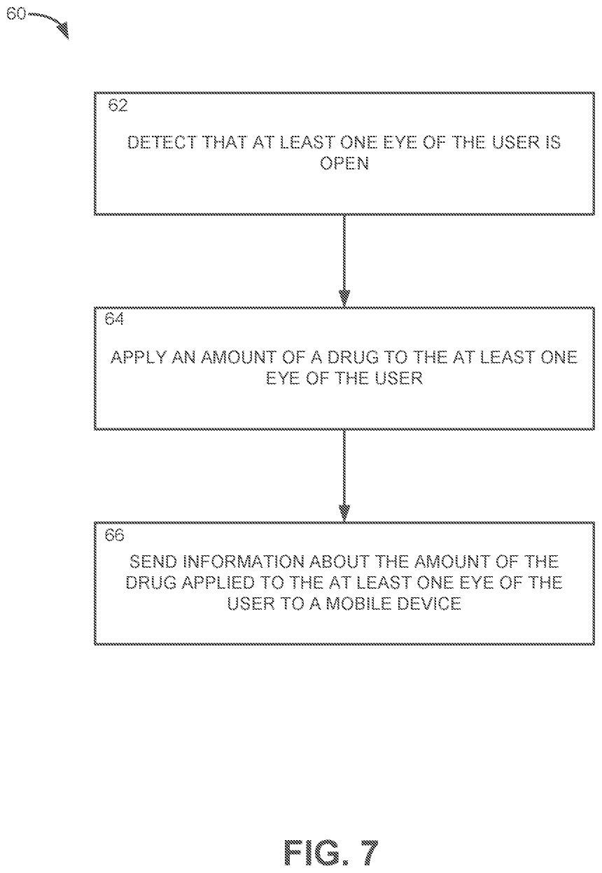

Another aspect of the present disclosure can include an example method 60, 70, and 90 (shown in FIGS. 7, 8, and 9) for efficient delivery of a drug to the eye using a smart head-mounted drug delivery device (examples of which are shown in FIGS. 1-6). The methods 60, 70, and 90 are illustrated as process flow diagrams with flowchart illustrations that can be implemented by one or more components of the smart head-mounted drug delivery device shown in FIGS. 1-6.

For purposes of simplicity, the methods 60, 70, and 90 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 60, 70, and 90. It should be noted that one or more steps of the methods 60, 70, and 90 can be executed by a hardware processor.

FIG. 7 is a process flow diagram of an example method 60 for tracking a user's use of a drug that is administered to the eye (e.g., a drug traditionally administered as eyedrops) to monitor and increase adherence to a prescription or instruction for use. At 62, an iris detection and tracking module of a head-mounted drug delivery system can detect that at least one eye of the user is open (e.g., the eyelids are open). If the eyelids are closed, then the iris detection and tracking module can continue monitoring until the eyelids are open. For example, the iris detection and tracking module can determine a blink rate of the eye. At 64, a drug delivery module of the head-mounted drug delivery system, can apply an amount of a drug to the at least one eye of the user while the eye is open. The amount can be predefined based on a prescription and/or instructions for use and may be programmable via a mobile device in communication with the head-mounted drug-delivery system. At 66, a wireless transmitter of the head mounted drug delivery system can send information about the drug applied to the at least one eye of the user to a mobile device (e.g., the mobile device of the user and/or the mobile device of a medical professional associated with the user). For example, the mobile device can log the amount of the drug applied to the at least one eye of the user at the time and keep a record over an extended period of time (e.g., a week, a month, a year, or the like). Information about the amount of the drug applied to the at least one eye (and the time of application) can be used to determine if a treatment is or is not working and/or if the user is being compliant with a prescription and/or instructions for use.

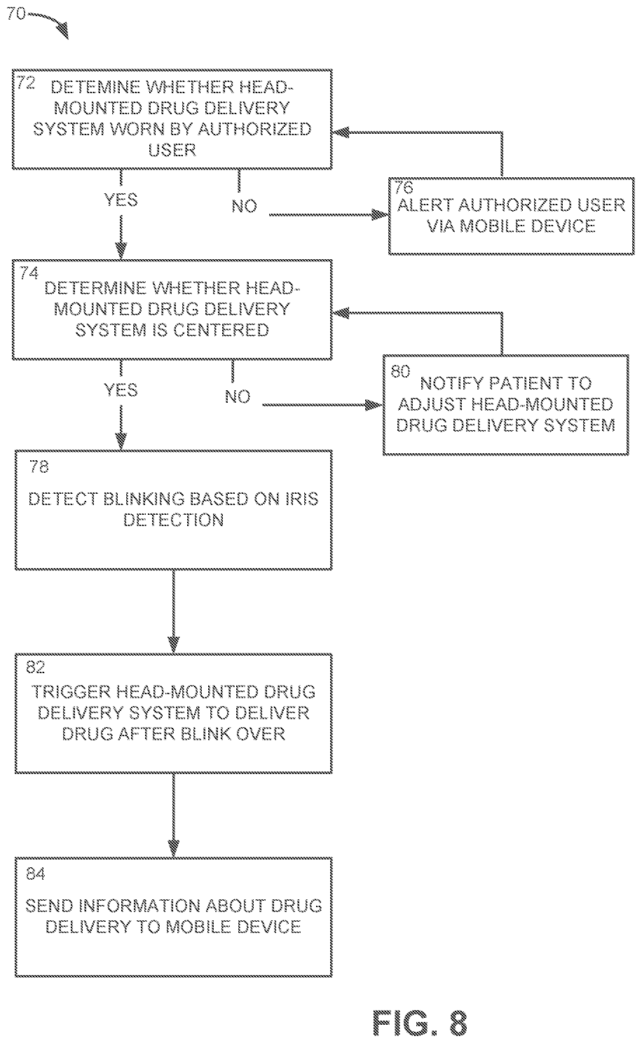

FIG. 8 is an example method 70 for delivery of a drug to at least one eye of a user of a head-mounted drug delivery system (e.g., a drug traditionally administered as eyedrops). At 72, it can be determined whether a head-mounted drug delivery system is worn by an authorized user. For example, the iris tracking and detection module can perform iris recognition based on a previous scan (e.g., as part of initial start-up process) of an authorized user. If the head-mounted drug delivery system is determined not to be worn by an authorized user, at 76, the authorized user can be alerted to wear the head-mounted drug delivery system (e.g., by a mobile device with a visual, tactile and/or audio alert). The mobile device can display a message instructing the user to wear the head mounted drug delivery system, for example. If the head-mounted drug delivery system is determined to be worn by the authorized user, at 74, it is then determined whether the-head mounted drug delivery system is centered on the user's face (e.g., worn correctly for most efficient drug delivery). If the head-mounted drug delivery system is determined not to be centered (worn correctly), at 80, the authorized user can be notified to center the head mounted drug delivery system (e.g., by a visual, tactile and/or audio notification via the mobile device). For example, the mobile device can display a message instructing the user to center the head-mounted drug delivery system (or to otherwise adjust the head mounted drug delivery system as necessary for proper functioning). The head-mounted drug delivery system can be centered over the eye, for example, using pupillometry where the camera can be used to help the user adjust the position or the drug delivery device to a position relative to the center of the eye by giving feedback (e.g., on an application being run on and displayed on the mobile device). If the head mounted drug delivery system is determined to be centered then, at 78, blinking can be detected (e.g., by the iris detection module of the head mounted drug delivery system). The blinking can be fast blinking.

At 82, the drug delivery system of the head-mounted drug delivery system can be triggered to deliver the drug (e.g., a predetermined amount of the drug) after the blink (or fast blink) is over. The triggering can be via a signal sent from the iris detection and tracking module to the drug delivery module. For example, when the iris detection and tracking module as determined the length of time the at least one eye is open after a blink a signal can be sent, or when a fast blink is detected. At 84, information about the drug delivery can be sent to a mobile device (e.g., for patient use, doctor use, or the like). The mobile device can aggregate the data for presentation and present (e.g., audio and/or visually) the data for the delivery or for a plurality of deliveries.

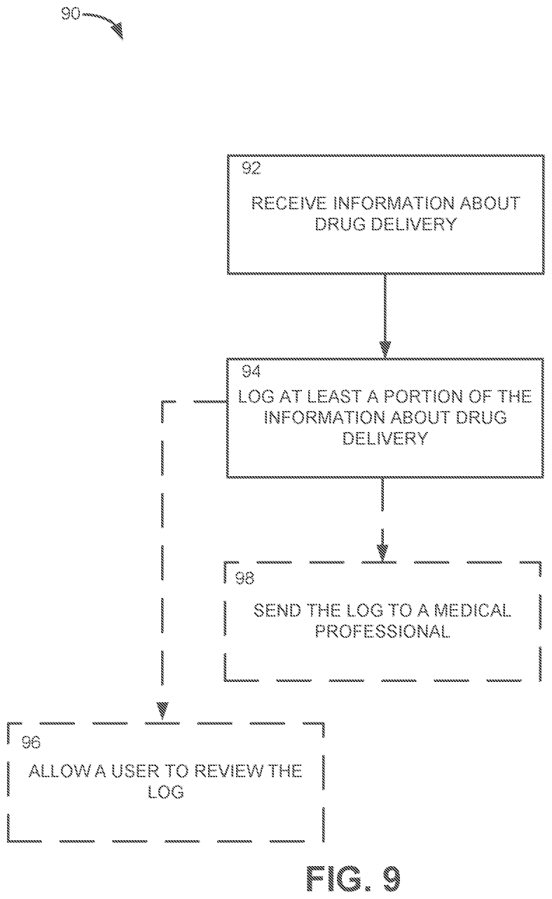

FIG. 9 is an example method 90 for logging a user's application of a drug to an eye (e.g., a drug traditionally administered as eyedrops). In some instances, the method 90 can be performed by a mobile device. In other instances, the method 90 can be performed by a head-mounted drug delivery system and/or a component associated with the head-mounted drug delivery system. In some instances, the method 90 can be performed in part by a mobile device and in part by a head-mounted drug delivery system (or an associated component thereof). At 92, information about the drug delivery to the at least one eye of the user via the drug delivery device can be received (e.g., by the mobile device, the head mounted drug delivery system, the component associated with the head-mounted drug delivery system, or the like). The information can be received, for example, after the drug has been delivered, after a notification or alert has been issued to the user, after the drug delivery has failed, or the like. For instance, the information can be received if the drug delivery has failed due to the authorized user not wearing the head-mounted drug delivery system at a scheduled or automatically determined delivery time, or the head-mounted drug delivery system being worn incorrectly, etc. As another example, if the drug delivery has completed successfully, the information (including at least one of the time, the length of time of the delivery, the amount of drug delivered, the amount of drug remaining, and any other relevant information) can be received. At 94, at least a portion of the information about the drug delivery can be logged (e.g., by the device receiving the information). The logged information can be stored locally in memory or in a database on cloud or server. The logged information can be used to determine statistics about the drug delivery over an extended period of time. Optionally, at 96, a user can be allowed to review the log (e.g., to see his success rate at delivery of the drug into the eye using the medical device) and/or at 98, the log can be sent to a medical professional (e.g., to determine whether a treatment is or is not working, whether the user is being compliant, whether the device is functioning properly, or the like). For example, the log can be sent from the mobile device to a mobile (or stationary) device of the medical professional. An alert (visual, tactile, and/or audio) can be sent to the patient when the patient is deemed to be non-compliant.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

What is claimed is:

1. A system comprising:
a head mounted drug delivery system comprising:
an iris detection and tracking module configured to detect an iris of at least one eye of a user;
a drug delivery module configured to receive at least one signal from the iris detection and tracking module and to release a drug into the user's eye, wherein the drug delivery module comprises:
a reservoir configured to store the drug, wherein the reservoir is at least one of refillable and removable;
a pump configured to propel an amount of the drug from the reservoir to at least one nozzle; and
the at least one nozzle configured to spray the amount of the drug onto the user's eye; and
a transmitter configured to communicate data wirelessly from the iris detection and tracking module and/or the drug delivery module; and
a mobile device configured to receive data from the iris detection and tracking module and/or the drug delivery module wirelessly.

2. The system of claim 1, wherein the head mounted drug delivery system further comprise a battery pack positioned on and/or in the frame and configured to power the iris detection and tracking module and/or the drug delivery module.

3. The system of claim 1, wherein the iris detection and tracking module is configured to detect presence, movement, location, and/or occlusion of the iris and comprises:
a microprocessor;
at least one light source configured to emit light towards at least one eye of the user; and
at least one detector configured to detect light reflected from the at least one eye of the user.

4. The system of claim 3, wherein the iris detection and tracking module is configured to determine whether the user is wearing the eyeglasses, whether the glasses are worn correctly, whether the user has blinked, and/or the user's blink speed.

5. The system of claim 1, wherein the iris detection and tracking module is configured to detect whether the user has blinked and send a signal to the drug delivery module to deliver the amount of the drug at a time after the user has blinked.

6. The system of claim 1, wherein the mobile device is configured to collect statistics related to the user's compliance with using the head mounted drug delivery system and/or drug delivery based on the data from the iris detection and tracking module and the drug delivery module.

7. The system of claim 1, wherein the iris detection and tracking module comprises a camera configured to detect whether the head mounted drug delivery system is worn by the user and the iris detection and tracking module is configured to authenticate the user wearing the head mounted drug delivery system based on the detection by the camera.

8. The system of claim 7, wherein the mobile device is configured to notify the user when the system determines the at least one of improper wearing of the head mounted drug delivery system or non-compliance.

9. The system of claim 7, wherein the camera is configured to detect dry eye and/or detect changes in intraocular pressure based on changes in a surface of the cornea.

10. The system of claim 1, wherein the head mounted drug delivery system comprises eyeglasses, wherein the at least one drug delivery nozzle comprises at least two drug delivery nozzles, each positioned on either side of a nose bridge of the eyeglasses and directed towards one of the eyes of the user.

11. The system of claim 1, wherein the at least one nozzle further comprises a drop absorption mechanism configured to sink any remaining droplets of the drug from at least one opening of the nozzle and back to the reservoir.

12. The system of claim 1, wherein the head mounted drug delivery system further comprise an ambient light sensor configured to enable the iris detection and tracking module to measure pupil size change of the at least one eye of the user.

13. Eyeglasses comprising:
an iris detection and tracking module, comprising a light source and/or a detector, configured to detect an iris of at least one eye of a user, wherein the iris detection and tracking module is at least partially positioned on a frame of the eyeglasses;
a drug delivery module coupled to the frame of the eyeglasses and configured to receive at least one signal from the iris detection module and tracking and to release a drug into the at least one eye of the user, wherein the drug delivery module comprises:
a reservoir configured to store the drug, wherein the reservoir is at least one of refillable and removable;
a pump configured to propel an amount of the drug from the reservoir to at least one nozzle; and
the at least one nozzle configured to spray the amount of the drug onto the at least one eye of the user.

14. The eyeglasses of claim 13, wherein data collected by the iris detection and tracking module is used to monitor at least one of eye detection, eye tracking, and blinking.

15. The eyeglasses of claim 13, wherein the light source and/or the camera detects the at least one eye of the user blinking and signals the drug delivery module to deliver the drug after the blinking.

16. A method comprising:
detecting, by an iris detection and tracking module of a head mounted drug delivery system, that at least one eye of the user is open;
applying, by a drug delivery module of the head mounted drug delivery system, an amount of a drug to the at least one eye of the user; and
sending, by a wireless transmitter of the head mounted drug delivery system, information about the drug applied to the at least one eye of the user to a mobile device,
wherein the mobile device logs the amount of the drug applied to the at least one eye of the user.

17. The method of claim 11, wherein the at least one eye is determined to be open a time after a blink is detected.

18. The method of claim 11, further comprising determining, by the iris detection and tracking module, whether the head mounted drug delivery system is being worn by the user, wherein when the head mounted drug delivery system is not determined to be worn, providing, via the mobile device, a message to the user to wear the head mounted drug delivery system.

19. The method of claim 11, further comprising determining, by the iris detection and tracking module, when a portion of the head mounted drug delivery system is centered over the at least one eye.

20. The method of claim 11, further comprising:
detecting, by the iris detection and tracking module, fast blinking; and
a time after the fast blinking, applying, by the drug delivery module, the amount of the drug to the at least one eye of the user.
